(19) United States
(12) Patent Application Publication
(10) Pub. No.: US 2018/0271541 A1
(43) Pub. Date: Sep. 27, 2018

(54) METHOD AND SYSTEM FOR APPLYING A CONSTRICTIVE FORCE TO A LIMB

(71) Applicant: Boa Technology Inc., Denver, CO (US)

(72) Inventors: John Figueiredo, Denver, CO (US); Bobby Dickensheets, Denver, CO (US); Stefan Sporrer, Denver, CO (US)

(73) Assignee: Boa Technology Inc., Denver, CO (US)

(21) Appl. No.: 15/934,590

(22) Filed: Mar. 23, 2018

Related U.S. Application Data
(60) Provisional application No. 62/476,229, filed on Mar. 24, 2017.

(51) Int. Cl. A61B 17/132

(52) U.S. Cl. CPC A61B 17/1327; A61B 2017/12004

(57) ABSTRACT

A device for applying a constrictive force about a limb includes a reel based device, a tension member, and a force application component. The reel based device includes a housing, a spool positioned within the housing, a load holding mechanism that enables the spool to rotate in a tightening direction within the housing and that prevents the spool from rotating in a loosening direction within the housing, and a knob that is coupled with the spool so that a rotation of the knob causes the spool to rotate in the tightening direction. The tension member is coupled with the spool so that as the spool rotates in the tightening direction, the tension member is wound about the spool. The force application component is coupled with the tension member so that as the tension member is wound about the spool, the force application component constricts about the limb.

### CROSS-REFERENCE TO RELATED APPLICATIONS

This application claims priority to Provisional U.S. Patent Application No. 62/476,229 filed Mar. 24, 2017, entitled “Method and System for Applying a Constrictive Force to a Limb,” the entire disclosure of which is hereby incorporated by reference, for all purposes, as if fully set forth herein.

### BACKGROUND

This invention relates generally to devices that are configured to applying a constrictive force to a limb and more specifically to a tourniquet system that includes a reel based closure device.

Tourniquet devices are often employed in times of emergency to apply a constrictive force to a limb and thereby impede or prevent the flow of blood to the limb. The use of such devices may be essentially when the limb is injured to the point that stopping blood flow is extremely difficult or impossible without applying the constrictive force. The use of the tourniquet devices may save the life of an individual or otherwise prevent catastrophic injury to the individual or limb. Tourniquet devices may be essential tools for individuals in certain professions such as emergency medical technicians (EMT), military personnel, and the like. Since tourniquet devices are often employed in emergency situations, having easy access to the tourniquet device and/or having a tourniquet device that is easy to manipulate and operate may result in the difference between life and death for an individual with significant injuries.

### BRIEF DESCRIPTION

The embodiments described herein are directed to reel based closure systems that may be used to apply a constrictive force or pressure to a limb. The constrictive force or pressure may be applied in order to prevent blood flow to the limb and thereby possibly save the life of an individual. According to one embodiment, a device for applying a constrictive force about a limb includes a reel based closure device, a tension member, and a force application component. The reel based closure device includes a housing having an interior region and a spool that is positioned within the interior region of the housing so that the spool is rotatable in a tightening direction within the housing and so that the spool is rotatable in a loosening direction within the housing. The reel based closure device also includes a load holding mechanism that is configured to enable the spool to rotate within the housing in the tightening direction and to prevent the spool from rotating in the loosening direction within the housing. The reel based closure device further includes a knob that is coupled with the housing and that is operationally coupled with the spool so that rotation of the knob relative to the housing causes the spool to rotate in the tightening direction within the housing. The is coupled with the spool so that as the spool is rotated in the tightening direction, the tension member is wound about the spool and so that as the spool is rotated in the loosening direction, the tension member is unwound from about the spool. The force application component is positionable about the limb and is coupled with the tension member so that as the tension member is wound about the spool, the force application component constricts about the limb and thereby applies the constrictive force to the limb.

In some embodiments, the force application component is a strap. In such embodiments, a distal end of the strap may be coupled with the tension member and a proximal end of the strap may be coupled with the housing of the reel based closure device. The tension member may be released from the distal end of the strap. The distal end of the strap may include a guide component that is configured to route or direct the tension member about the distal end of the strap.

In some embodiments, the force application component includes a guide that is configured to route or direct the tension member about the force application component. The force application component may also include a lock mechanism that engages with the tension member to prevent movement of the tension member relative to the force application component. The lock mechanism may be a pair of cam members. The load holding mechanism may be disengageable to enable the spool to rotate in the loosening direction.

According to another embodiment, a method of applying a constrictive force about a limb includes positioning a constrictive device about the limb in which the constrictive device includes a reel based closure device, a tension member, and a force application component. The reel based closure device includes a housing, a spool that is rotatable in a tightening direction relative to the housing and that is rotatable in a loosening direction relative to the housing, a load holding mechanism that enables the spool to rotate in the tightening direction and that prevents the spool from rotating in the loosening direction, and a knob that is operationally coupled with the spool so that rotation of the knob relative to the housing causes the spool to rotate in the tightening direction. The tension member is coupled with the spool so that as the spool is rotated in the tightening direction, the tension member is wound about the spool and so that as the spool is rotated in the loosening direction, the tension member is unwound from about the spool. The force application component is positionable about the limb and is coupled with the tension member so that as the tension member is wound about the spool, the force application component constricts about the limb. The method also includes rotating the knob to rotate the spool in the tightening direction and wind the tension member about the spool and thereby apply the constrictive force to the limb via the force application component. In some embodiments, the method further includes disengaging the load holding mechanism to enable the spool to rotate in the loosening direction.

In some embodiment, the force application component is a strap. In such embodiments, a distal end of the strap may be coupled with the tension member and a proximal end of the strap may be coupled with the housing of the reel based closure device. In such instances, the method may also include releasing the tension member from the distal end of the strap. The distal end of the strap may include a guide component that is configured to route or direct the tension member about the distal end of the strap.

In some embodiments, the force application component includes a guide that is configured to route or direct the tension member about the force application component. The force application component may also include a lock mechanism that engages with the tension member to prevent movement of the tension member relative to the force application component. The lock mechanism may be or include a pair of cam members.

### BRIEF DESCRIPTION OF THE DRAWINGS

The present invention is described in conjunction with the appended figures:

FIGS. 1a-c illustrate a reel based closure device that may be used as a tourniquet to provide a constrictive force to a limb.

FIGS. 2a-c illustrate an alternative reel based closure device that may be used to provide a constrictive force to a limb.

FIGS. 3a-4c illustrate alternative reel based closure systems that may be used to provide a constrictive force to a limb.

FIGS. 5a-b illustrate a tourniquet device in the form of a watch that may be used to provide a constrictive force to a limb.

FIGS. 6a-b illustrate a tourniquet device in the form of a band that may be worn about a person's body and that may be used to provide a constrictive force to a limb.

FIGS. 7a-c illustrate an alternative tourniquet device in the form of a band that may be worn about a person's body and that may be used to provide a constrictive force to a limb.

FIG. 8 illustrates a tourniquet device in which the device is concealed within an individual's jacket or shirt.

FIG. 9 illustrates multiple tourniquet devices positioned about a user's body and concealed by one or more articles of clothing.

FIGS. 10a-b illustrate a tourniquet device that includes a strap body and a reel based closure device.

FIGS. 11a-b illustrate a tourniquet device that is configured for placement around relatively large limbs.

FIGS. 12a-b illustrate a tourniquet device that includes a strap body and a reel based closure device in which the strap body includes a hole through which the reel based closure device is inserted.

FIGS. 13a-b illustrate tourniquet devices that are configured to enable macro adjustment of a strap body about a limb prior to operating a reel based closure device to tighten the strap body about the limb.

FIGS. 14a-c illustrate variations of reel based closure devices, tension members, and pad members that may be employed in the tourniquet system of FIG. 13b.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION OF THE DRAWINGS

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth in the appended claims.

The embodiments described herein provide embodiments of reel based closure systems and components of such systems that may be used to constrict or provide pressure to a limb. In some instances, the reel based closure systems and components may be used as a tourniquet in order to prevent blood flow to the limb and thereby possibly save the life of an individual. These systems may be ideal for emergency medical technicians (EMT) or military personnel, which need a quick and effective system for stopping or minimizing blood flow to a portion of the body.

The reel based closure systems typically include a tension member, such as a lace or strap, that is relatively strong and flexible and may be ideal in applying the constrictive force to the limb. In some instances, the reel based closure system may include a guide member that is used to route or direct the lace. The reel based closure system typically includes a knob that may be grasped and rotated by a user to tension the tension member. Exemplary embodiments of reel based closure devices are further described in U.S. patent application Ser. No. 13/098,276, filed Apr. 29, 2011, titled “Reel Based Lacing System”, U.S. patent application Ser. No. 14/328,521, filed Jul. 10, 2014, titled “Closure Devices Including Incremental Release Mechanisms and Methods Therefor,” and U.S. patent application Ser. No. 12/623,362, filed Nov. 20, 2009, titled “Reel Based Lacing System”, the entire disclosures of which are incorporated by reference herein.

In other embodiments, the tightening mechanism may be a motorized device or mechanism that tensions the tension member or lace. An exemplary embodiment of a motorized mechanism that may be used to tension the lace is further described in U.S. patent application Ser. No. 14/015,807, filed Aug. 30, 2013, titled “Motorized Tensioning System for Medical Braces and Devices”, the entire disclosure of which is incorporated by reference herein.

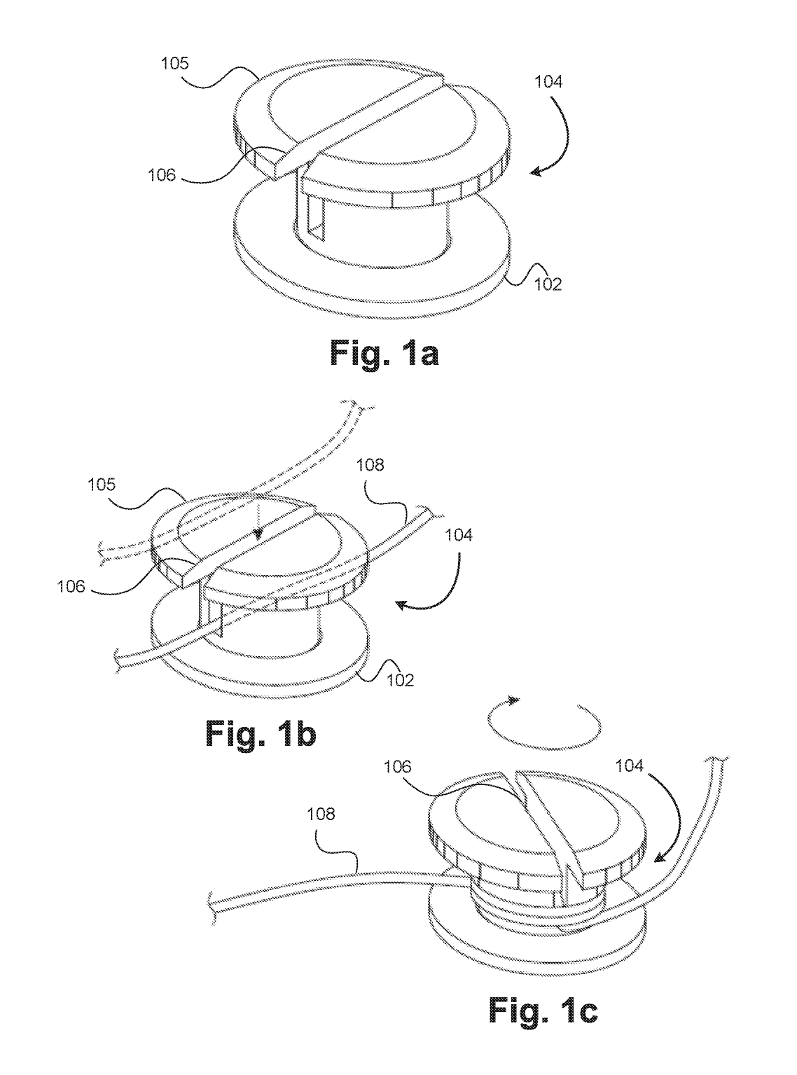

Referring now to FIGS. 1a-c, illustrated is a reel based closure device 102 that may be used as a tourniquet to provide a constrictive force to a limb. The reel based closure device 102 includes an upper knob member or flange 105 that may be grasped and rotated by a user. The reel based closure device 102 also includes a channel 104 about which a lace 108 is wound as the knob member 105 is grasped and rotated by a user. A slot 106 is defined in the reel based closure device 102 that extends axially downward from an upper surface of the knob member 105 to within the channel 104. As shown in FIG. 1b, the slot 106 is configured so that the lace 108 may be positioned within the slot 106 and radially extend from channel 104 on opposite sides of the reel based closure device 102. In some embodiments, the slot 106 may be angled so that the slot narrows as the slot 106 extends axially downward from the upper surface of the knob member 105. The narrowing slot 106 may increase the frictional engagement between the lace 108 and slot 106 as the lace 108 is inserted within the slot 106. As shown in FIG. 1c, with the lace 108 disposed within the slot 106, rotation of the reel based closure device 102 via the knob member 105 causes the lace 108 to be wound around the channel 104. As illustrated in other figures of the instant application, winding of the lace 108 about the channel 104 may cause the lace 108 to constrict about a limb.

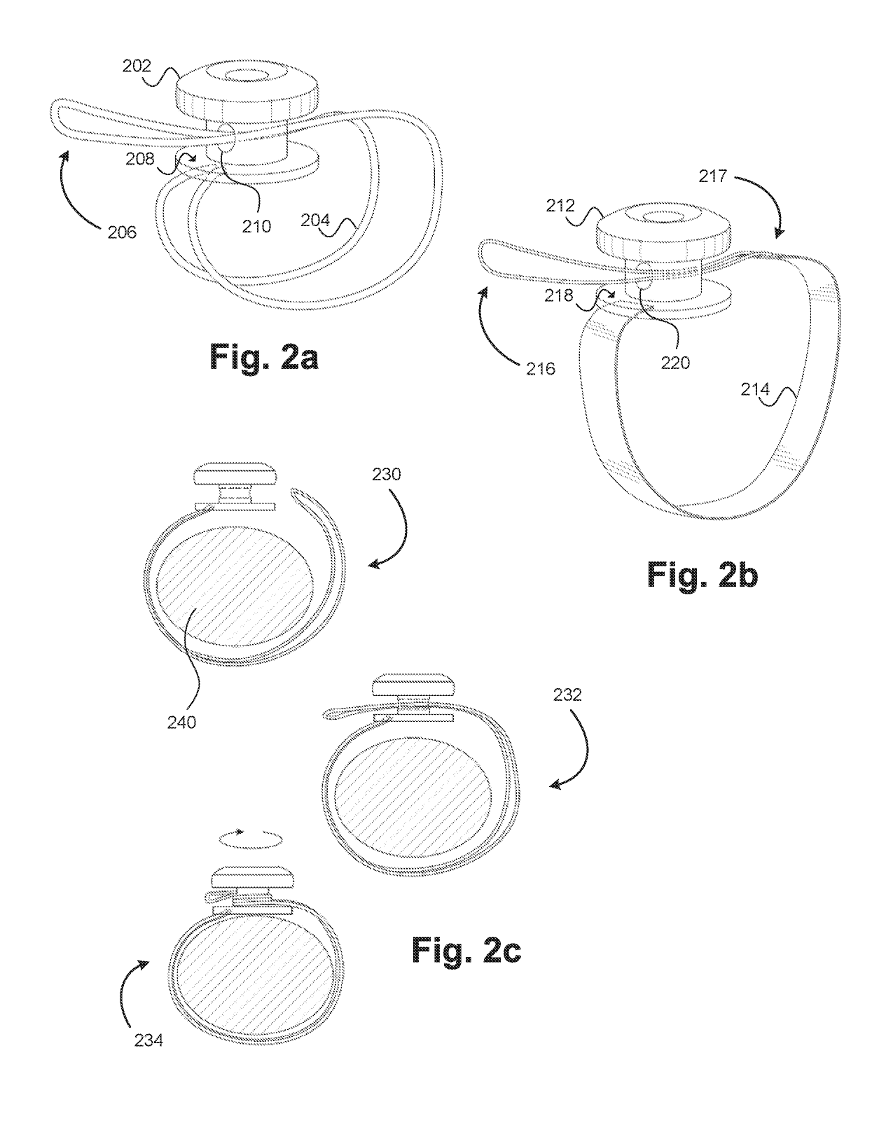

Referring now to FIGS. 2a-c, illustrated are alternative embodiments of a tourniquet device. FIG. 2a illustrates a reel based closure device 202 that includes a knob member and channel that are similar to the embodiment illustrated in FIGS. 1a-c. The reel based closure device 202 does not include a slot 106, but rather includes a channel or through hole 210 through which a loop of lace 206 is inserted. The loop of lace 206 is formed by attaching opposing ends 208 of the lace 204 to the reel based closure device 202, such as by attaching the opposing ends 208 to a bottom flange or surface of the reel based closure device 202. The loop of lace 206 may then be inserted through the channel 210 so that a portion of the lace 204 is disposed within the channel 210. With the lace 204 disposed within the channel 210, rotation of the reel based closure device 202 via the knob member causes the lace 204 to be wound about the reel based closure device's channel. In some instances, the reel based closure device 202 may include a feature, such as a hook, barb, or other feature, about which the loop of lace 206 may be positioned to couple the loop of lace 206 with the reel based closure device 202 and thereby prevent the loop of lace 206 from being pulled or retracted through the channel 210 as the reel based closure device 202 is rotated.

FIG. 2b illustrates a similar embodiment except that the reel based closure device 212 includes a strap member 214. A proximal end 218 of the strap member 214 is attached to the reel based closure device 212 while a distal end 217 of the strap member 214 is attached to a lace. The lace forms a loop 216 that is inserted through a channel 220 as described above. Rotation of the reel based closure device 212 causes the lace to be wound about the reel based closure device's channel, which causes the strap member 214 to be tensioned due to the coupling of the lace and strap member 214. FIG. 2c illustrates an operation of the reel based closure devices, 202 & 212, of FIGS. 2a and 2b, or of any of the other reel based closure devices described herein such as the reel based closure device 102 of FIGS. 1a-c. FIG. 2c illustrates that in a first step 230, the reel based closure device is positioned about a limb 240 so that the tension member (e.g., lace or strap member) encircles the limb 240. In step 232, the tension member is attached to the reel based closure device, such as by inserting a loop of lace through a channel, positioning a portion of the lace through a slot, and the like. In some embodiments, step 232 is not needed since the lace is already attached to the reel based closure device. In step 234, the reel based closure device is operated to tighten the tension member about the limb 240 and thereby provide a constrictive force to the limb 240.

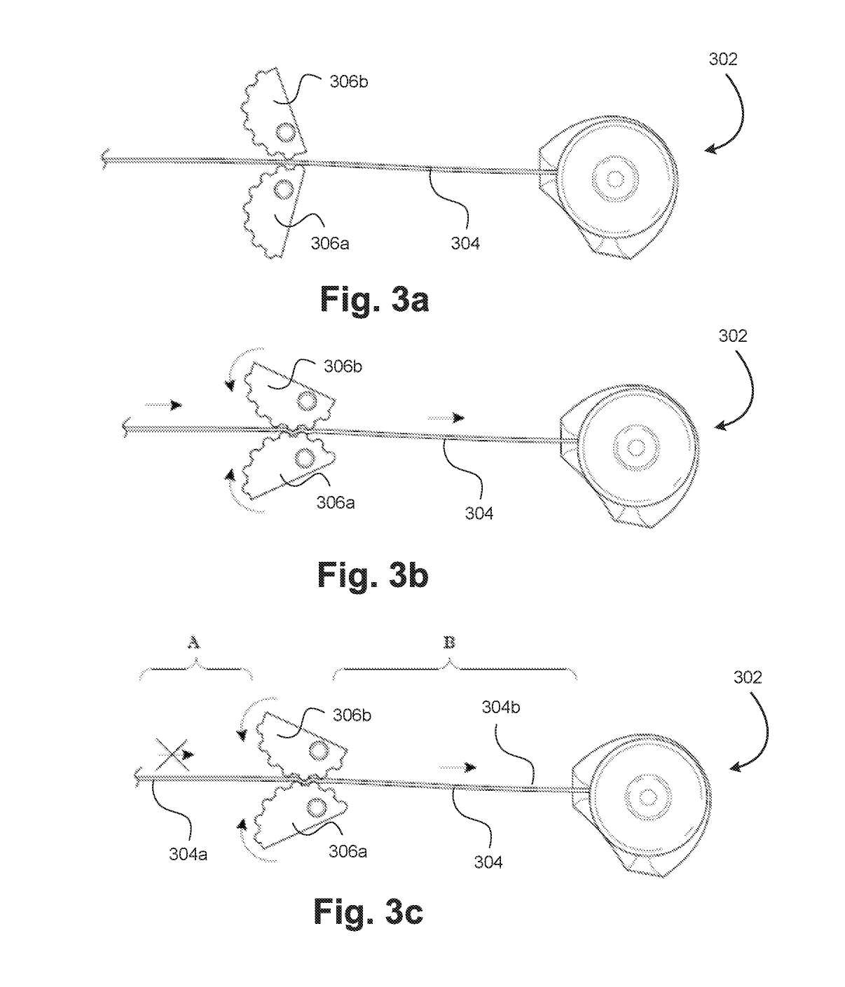

Referring now to FIGS. 3a-c, illustrated is another reel based closure device 302 that may be employed as a tourniquet to stop blood flow to a limb, or that may be applied for some other purpose. The reel based closure device 302 includes a knob that is rotated to tension a lace 304 by retracting the lace 304 within a housing. The reel based closure device 302 employs a pair of interlocking cam members, 306a and 306b, that are configured to pivot into engagement and thereby prevent movement or sliding of the lace 304 relative to the cam members, 306a and 306b. FIG. 3a illustrates the interlocking cam members, 306a and 306b, in a disengaged state in which the interlocking cam members, 306a and 306b, do not lockingly engage with the lace 304. FIG. 3b illustrates that as the reel based closure device 302 is operated, the lace 304 is retracted toward the reel based closure device 302, which causes the interlocking cam members, 306a and 306b, to pivot into engagement with the one another and with the lace 304. Specifically, the lace 304 may be positioned between the interlocking cam members, 306a and 306b, so that a minimal frictional engagement occurs between the lace 304 and the interlocking cam members, 306a and 306b. The frictional engagement may be sufficient so that sliding or movement of the lace 304 relative to the interlocking cam members, 306a and 306b, causes the cam members to pivot into the engaged position. The interlocking cam members, 306a and 306b, and/or lace 304 may be designed so that a desired amount of sliding or retraction of the lace 304 occurs before the interlocking cam members, 306a and 306b, pivot into engagement.

FIG. 3c illustrates that after the interlocking cam members, 306a and 306b, are engaged, a portion of the lace 304a that is disposed distally of the interlocking cam members, 306a and 306b, is prevented from movement toward the reel based closure device 302 due to the engagement of the interlocking cam members, 306a and 306b, and the lace 304. Since the distally positioned lace 304a is prevented from further movement, a length of lace A remains constant. Further operation of the reel based closure device 302 may cause the closure device to move or migrate toward the interlocking cam members, 306a and 306b, which will reduce a length of lace B between the closure device and the interlocking cam members. The system of FIGS. 3a-c may be ideally suited for situations where it is desired to maintain a length of the distal lace A, such as when the distal lace A is positioned over sensitive tissue of a limb or for various other reasons.

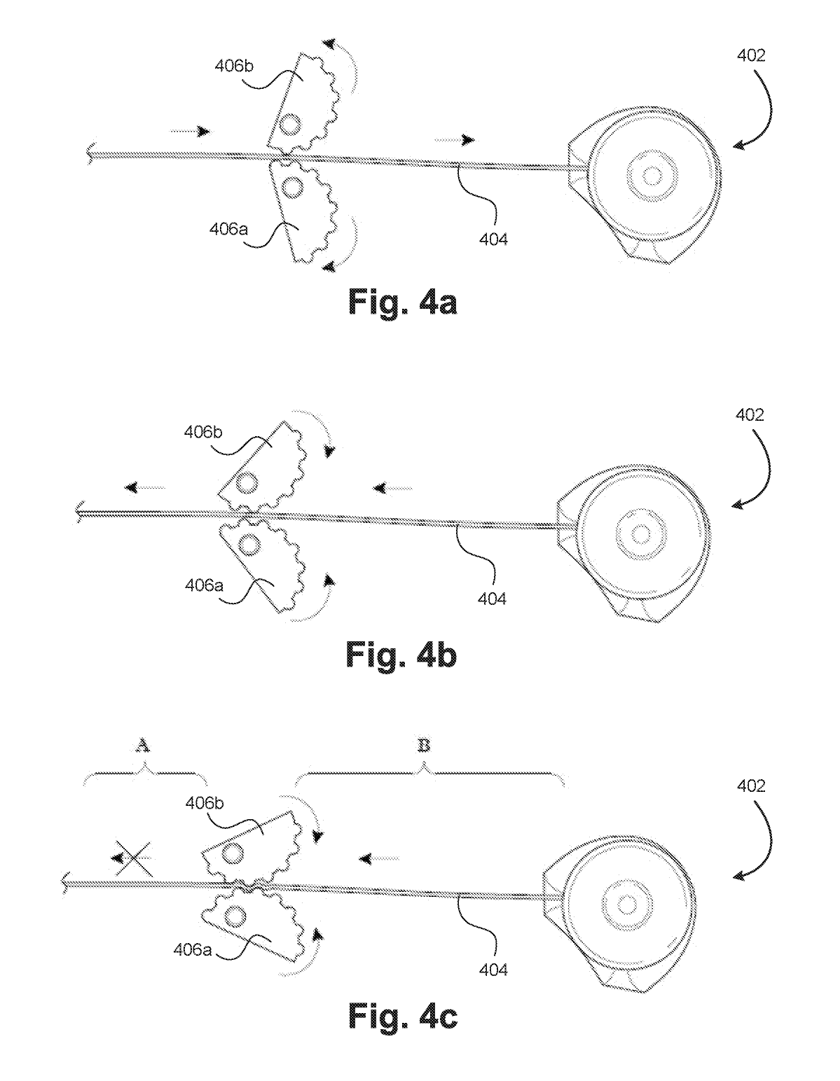

Referring now to FIGS. 4a-c, illustrated is another reel based closure device 402 that employ a pair of interlocking cam members, 406a and 406b, that pivot into engagement and prevent movement or sliding of a lace 404 relative to the cam members. FIG. 4a illustrates the interlocking cam members, 406a and 406b, in a disengaged state in which the interlocking cam members, 406a and 406b, do not engage with the lace 404. Unlike the interlocking cam members, 306a and 306b, of FIGS. 3a-c, the interlocking cam members, 406a and 406b, of FIGS. 4a-c are positioned to pivot in an opposite direction, which allows the lace 404 to be retracted toward the reel based closure device 402 and through the interlocking cam members, 406a and 406b. The interlocking cam members, 406a and 406b, are configured to pivot into engagement when the lace 404 move or slides in an opposite direction away from the reel based closure device 402 as illustrated in FIG. 4b, which prevents the length of lace A in the distal portion of the lace 404 from increasing or lengthening. The interlocking cam members, 406a and 406b, may be employed to maintain the lace 404 in a constricted or tensioned state about a limb and may primarily function to reinforce the reel based closure device 402, or to function as a backup mechanism in case the reel based closure device 402 fails or is accidently operated to loosen the lace tension. Specifically, the tourniquet system of FIGS. 4a-c may be positioned about a limb and the reel based closure device 402 may be operated to apply a constrictive force to the limb. The interlocking cam members, 406a and 406b, may then engage to prevent the lace 404 from moving relative to the limb and thereby loosening the applied constrictive force.

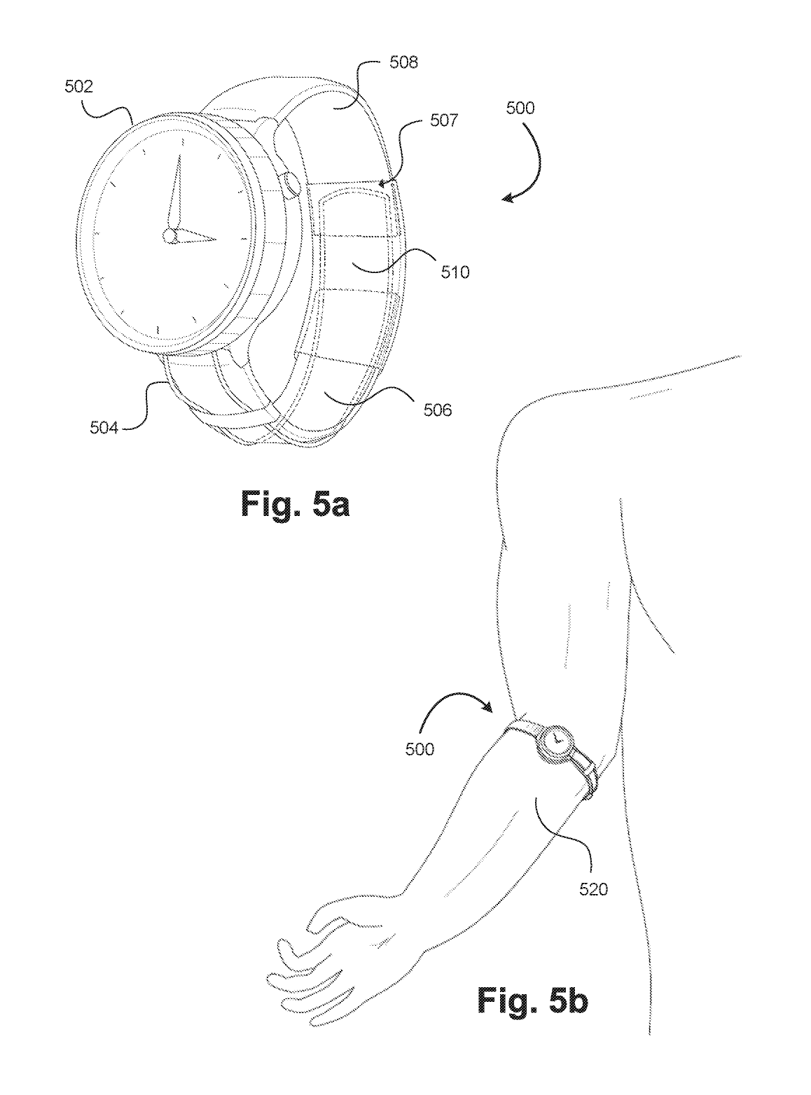

Referring now to FIGS. 5a-b, illustrated is another embodiment of a tourniquet device 500 that may be employed to provide a constrictive force to a limb. The tourniquet device 500 is in the form of a wrist watch and thus, may serve the dual purpose of being capable of providing the constrictive force while being an aesthetic piece of apparel. The tourniquet device 500 may allow a user, such as an emergency medical technician (EMT), to carry the tourniquet device on their person in a manner that is not distractive or otherwise obvious.

The tourniquet device 500 includes a watch face 502, which functions as the knob of a reel based closure device. The watch face 502 is operably coupled with lace 504 so that rotation of the watch face 502 causes the lace 504 to be tensioned. A distal portion 507 of the lace 504 is attached to a strap or band 508 of the watch. A proximal portion of the lace 504 is typically positioned over an opposite strap or band 506 of the watch in a manner such that the lace 504 slides or move relative to the strap or band 506. Tensioning of the lace 504 causes the two bands, 506 and 508, of the watch to be drawn together, which causes the band, 506 and 508, to constrict about a limb. A portion of the lace 504, or the entire lace 504, may be positioned within or under a tunnel or channel 510 so that it is hidden from view, which may enhance the visual appeal of the watch. FIG. 5b illustrates the tourniquet device 500 positioned about a user's forearm 520 in a manner that would allow the tourniquet device 500 to be operated to apply the constrictive force. The tourniquet device 500 is typically configured so that the diameter of the watch bands, 506 and 508, may be greatly increased to enable the tourniquet device 500 to be fit about larger limbs, such as the leg.

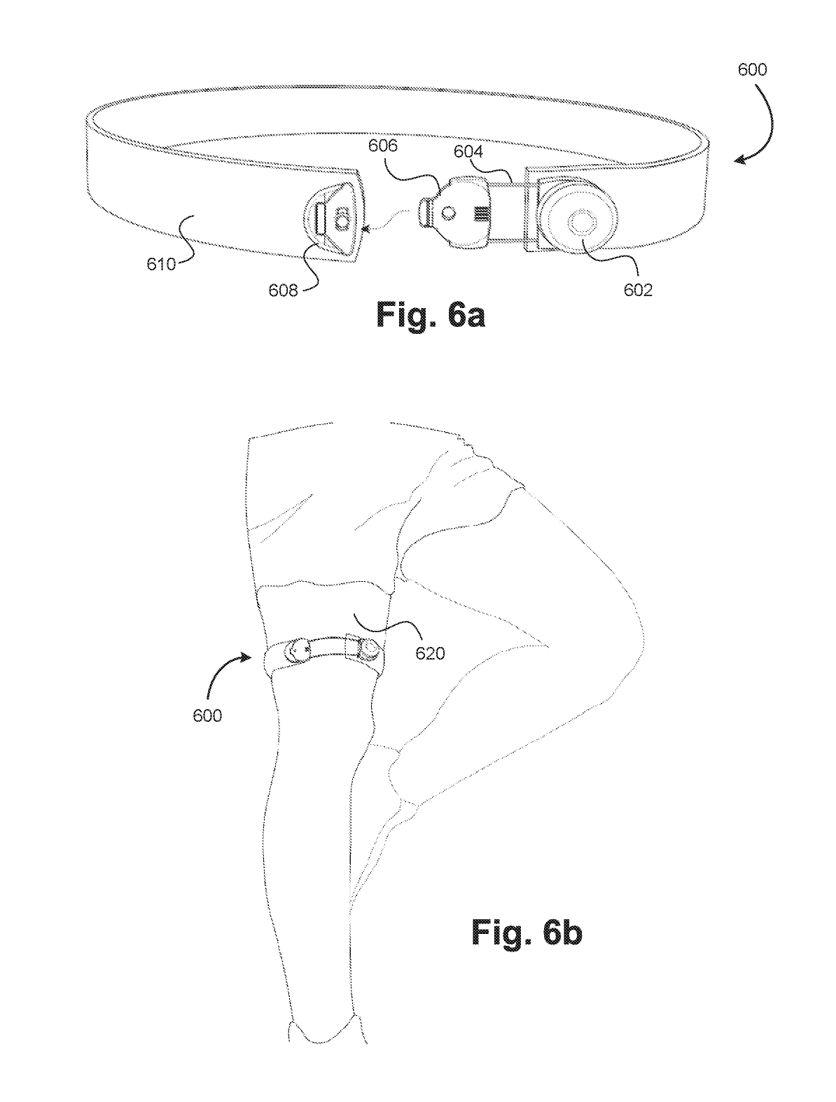

Referring now to FIGS. 6a-b, illustrated is another embodiment of a tourniquet device 600 that may be employed to provide a constrictive force to a limb. The tourniquet device 600 is in the form of a band that may be worn as an aesthetic piece of apparel and/or easily positioned and carried on a person's body. The tourniquet device 600 includes a strap or band body 610 and a reel based closure device 602 that is attached to one end of the strap body 610. A lace 604 is operably coupled with the reel based closure device 602 so that rotation of the reel based closure device's knob causes the lace 604 to be tensioned. The lace 604 is operably attached to a male coupling component 606 that may be releasably attached or coupled with a female coupling component 608 that is attached to the opposite end of the strap body 610. When the male and female coupling components, 606 and 608, are attached, tensioning of the lace 604 via the reel based closure device causes a diameter of the strap body 610 to be reduced. An exemplary embodiment of male and female coupling components is described in U.S. Pat. No. 9,516,923, entitled “Coupling Members for Closure Devices and Systems,” the entire disclosure of which is incorporated by reference herein.

FIG. 6b illustrates that the tourniquet device 600 may be positioned or attached about a limb 620 by wrapping the strap body 610 around the limb 620 and coupling the male and female coupling components, 606 and 608, together. The reel based closure device 602 may then be operated to constrict the strap body 610 about the limb 620 and thereby provide a desired constrictive force to the limb.

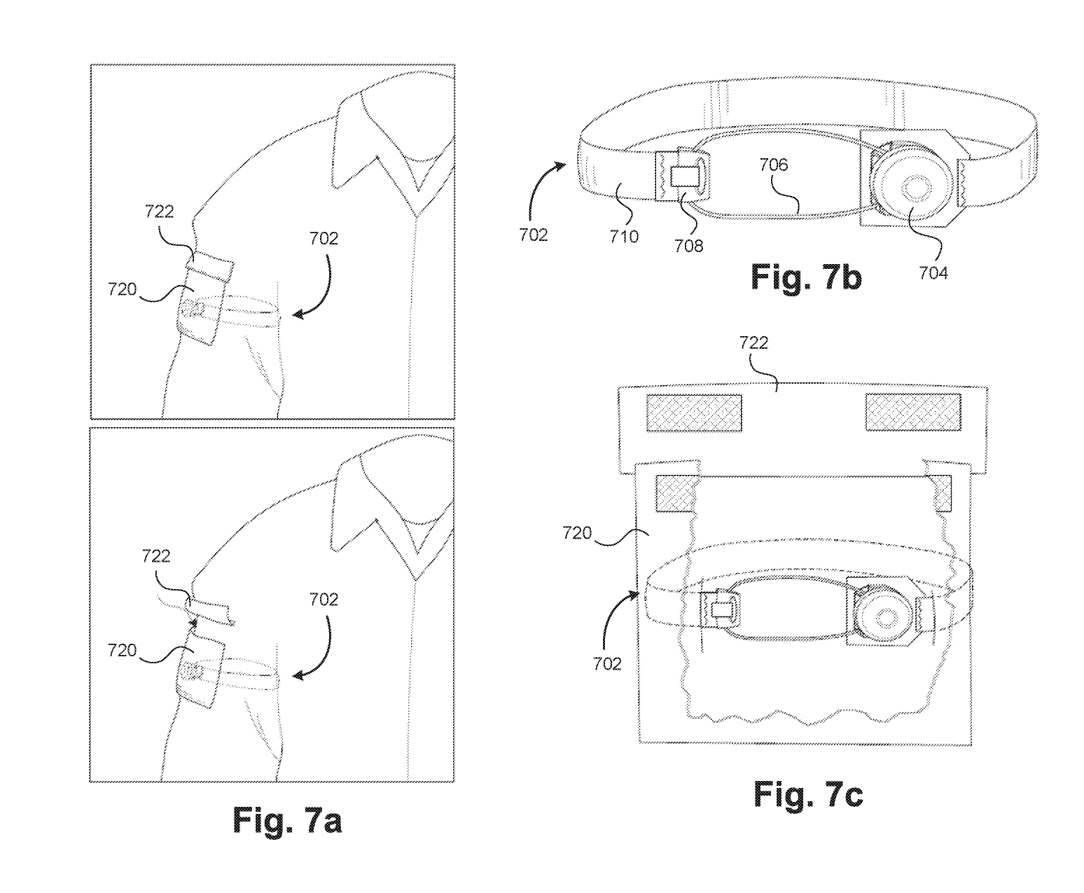

Referring now to FIGS. 7a-c, illustrated is another embodiment of a tourniquet device 702, which may be ideal for single hand use. As illustrated in FIG. 7b, the tourniquet device 702 is similar to that illustrated in FIGS. 6a-b except that the male and female coupling component of FIGS. 6a-b are not employed. Rather, the tourniquet device 702 includes a different guide member 708 that is attached to one end of a strap body 710 and that functions to guide or redirect the lace 706 toward a reel based closure device 704 that is attached to the opposite end of the strap body 710. The guide member 708 may or may not be configured to release or uncouple from the lace 706.

FIG. 7a illustrates that a jacket or shirt may be configured to carry and conceal the tourniquet device 702. Specifically, the jacket or shirt includes a pocket 720 within which the tourniquet device 702 is positioned. A material flap 722 may be foldable over the pocket 720 to encase the tourniquet device 702 within the pocket. FIG. 7c illustrates the pocket 720 and material flap 722 in greater detail. The pocket 720 and material flap 722 include attachment features (e.g., hook and loop fasteners, mechanical buttons or snaps, etc.) that allow the material flap 722 to be secured over the pocket 720. In some instances, the jacket and pocket 720 may be configured so that the tourniquet device 702 is positioned around the user's limb as the jacket is worn. The pocket 720 may allow access to the tourniquet device 702 to enable the user (e.g., an EMT, military personnel, etc.) to readily access the tourniquet device 702 and remove it from the pocket and jacket. The ease of access is ideally suited for instances where the tourniquet system is immediately needed, such as in response to an accident, in the middle of warfare, etc.

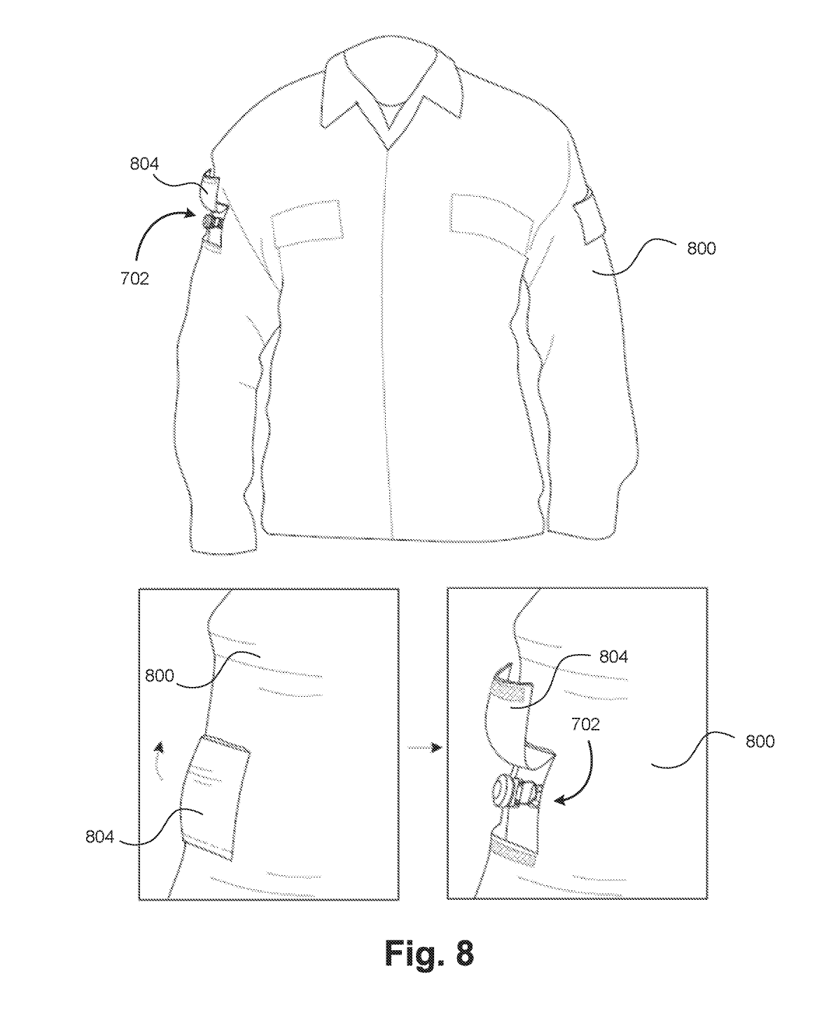

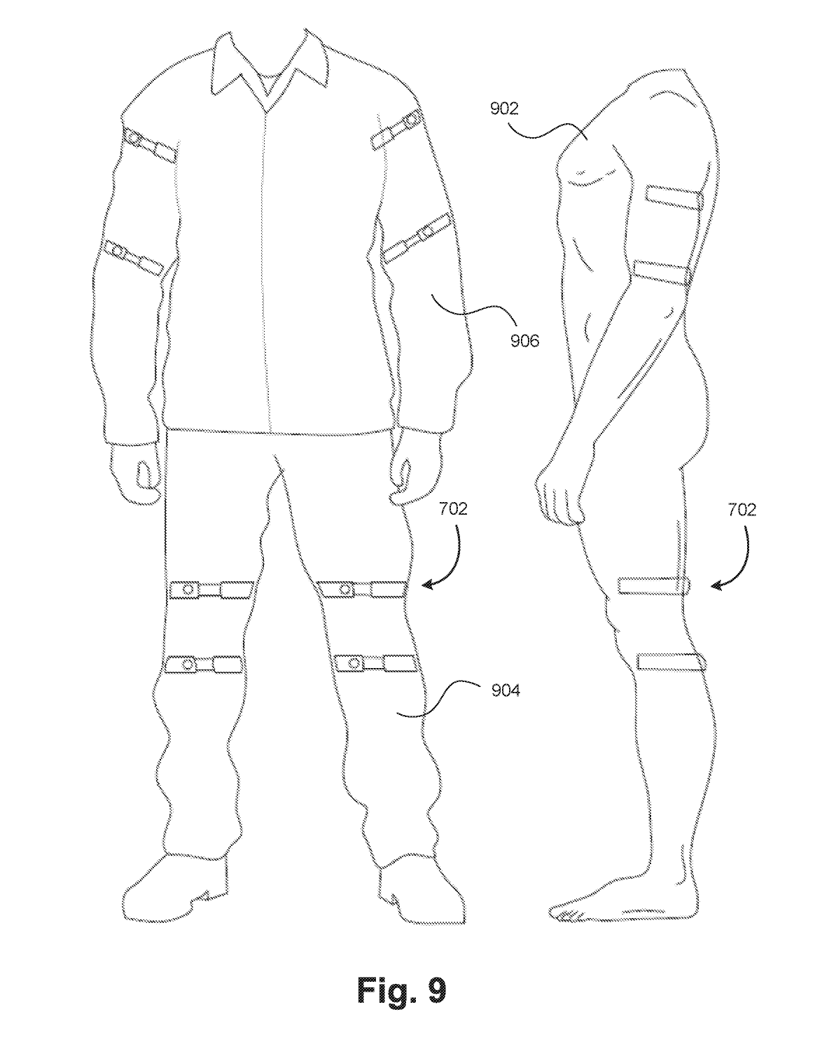

FIG. 8 illustrates another embodiment in which the tourniquet device 702 is concealed within a jacket or shirt 800. The jacket 800 does not include a pocket, but instead includes a window or open section that provides direct access to the tourniquet device 702. A material flap 804 is foldable over the window or open section in order to conceal the tourniquet device 702 within the jacket. In use, the tourniquet device 702 may be positioned around the user's limb (e.g., the upper arm) so that the tourniquet device 702 is positioned directly under the material flap 804 when the jacket 800 is worn. When the tourniquet device 702 is needed, the user may access the tourniquet device 702 via the material flap 804. The tourniquet device 702 may be configured to allow the lace to be detached from the strap to enable the tourniquet device 702 to be removed from about the user's limb. FIG. 9 illustrates that multiple tourniquet devices 702 may be positioned about a user's body 902 and concealed by one or more articles of clothing, 904 and 906. Each article of clothing, 904 and 906, may have a material access flap (not shown) that provides direct access to the tourniquet device 702. The tourniquet devices 702 may be positioned about the body 902 in areas that provide the least amount of discomfort as the tourniquet device 702 is worn. In addition, FIG. 9 illustrates strategic positions wherein tourniquet may be applied to a limb via one or more of the tourniquet devices described herein. Applying a tourniquet in the illustrated areas may limit tissue damage that results from applying the tourniquet.

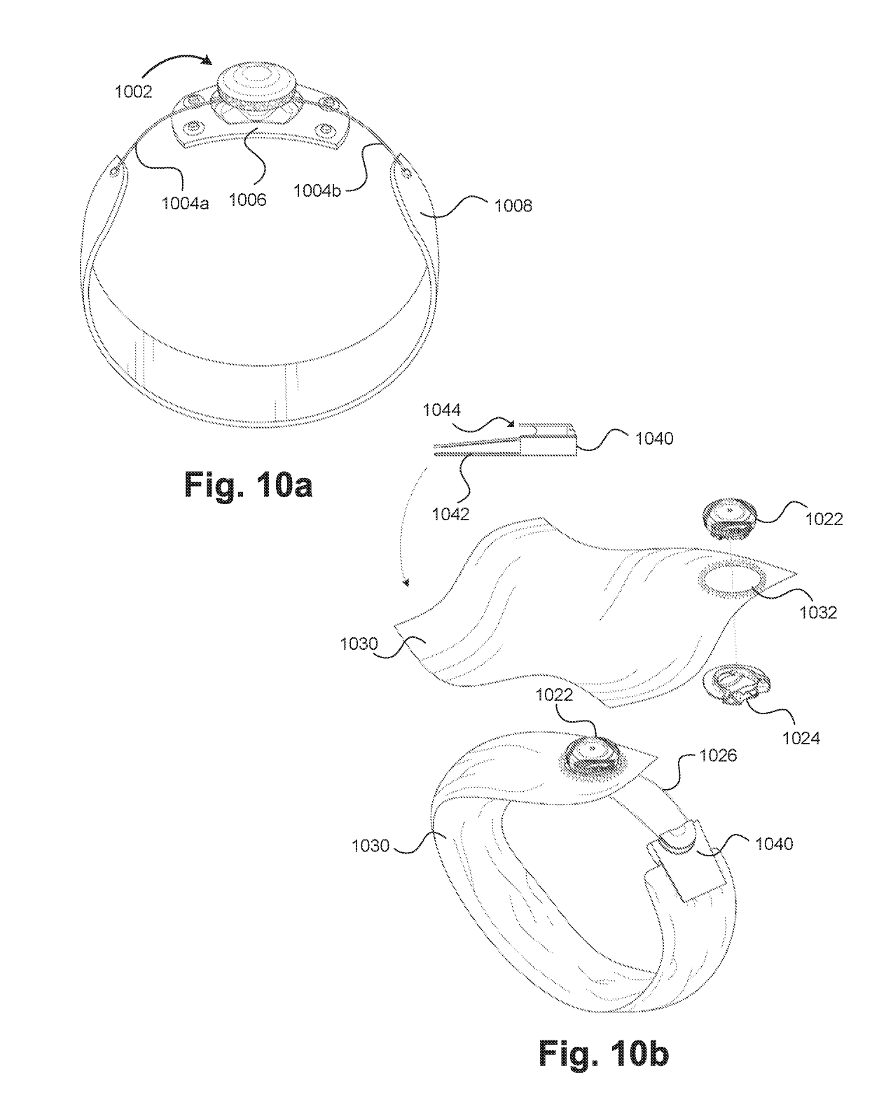

Referring now to FIGS. 10a-b, illustrated are other embodiments of tourniquet devices. FIG. 10a illustrates a device that includes a strap body 1008 and a reel based closure device 1002. The reel based closure device 1002 is operably coupled with a pair of laces, 1004a and 1004b, that are each in turn attached to opposing ends of the strap body 1008. The reel based closure device 1002 may be positioned on a pad member 1006 in order to distribute a radially inward force that is exerted on the limb as the tourniquet device is employed to deliver the constrictive force and/or worn by a user (e.g., EMT, military personnel, etc.). Stated differently, the pad member 1006 may minimize excessive pressure points that may cause extreme discomfort and/or puncture the skin. Tensioning of the pair of laces, 1004a and 1004b, causes the opposing ends of the strap body 1008 to move together, which when place about a limb, causes the strap body 1008 to constrict about the limb.

FIG. 10b illustrates components that may be used with a material body, such as a cloth material, in order to form a tourniquet system. The components are specifically designed so that they may be used with the material body to form the tourniquet system with little alteration to the material body. For example, a material body 1030, such as a piece of clothing, may be cut or formed into a rectangular or other shaped object and a hole 1032 may be cut into the material body 1030. A guide member 1040 may then be attached to one side or end of the material body 1030, typically opposite to where the hole 1032 is positioned. The guide member 1040 includes attachment features 1042 that enable the guide member 1040 to be quickly and easily attached to the material body 1030. In the illustrated example, the guide member 1040 includes angled teeth 1042 that are positioned along opposing inner surfaces of a channel. The angled teeth 1042 enable insertion of the material body 1030 within the channel of the guide member 1040 and prevent retraction of the material body 1030 relative thereto by biting or gripping into the material body 1030. The guide member 1040 includes an open channel 1044 that is configured to enable a lace 1026 to be positioned around and within the channel 1044.

A reel based closure device 1022 is attachable to the material body 1030 by positioning a bottom end of the reel based closure device 1022 through the hole 1032 and by attaching the bottom end of the reel based closure device 1022 with a base member 1024. The base member 1024 is sized larger than the hole 1032 so that when the reel based closure device 1022 and the base member 1024 are attached, the reel based closure device 1022 and base member 1024 may not be pulled or extracted through the hole 1032. Likewise, the reel based closure device 1022 is slightly larger than the hole 1032 to prevent the reel based closure device 1022 from being pulled through the hole 1032. The reel based closure device 1022 is typically attached to an opposite end of the material body 1030 relative to the guide member 1040.

With the guide member 1040 and the reel based closure device 1022 attached to the material body 1030, the material body may then be wrapped around a user's limb. The lace 1026, which is pre-attached to the reel based closure device 1022, may then be released from the reel based closure device 1022 and positioned within the channel 1044 of the guide member 1040 in order to form a tourniquet system that fully encircles the limb. The reel based closure device 1022 may then be operated to tension the lace 1026, which causes the material body 1030 to constrict about the limb. The material body 1030 may be formed from an article of clothing, such as a jacket, shirt, pants, etc. of the user. In other instances, the material body 1030 may be formed from material that is nearby, such as a towel, rag, tubing, etc.

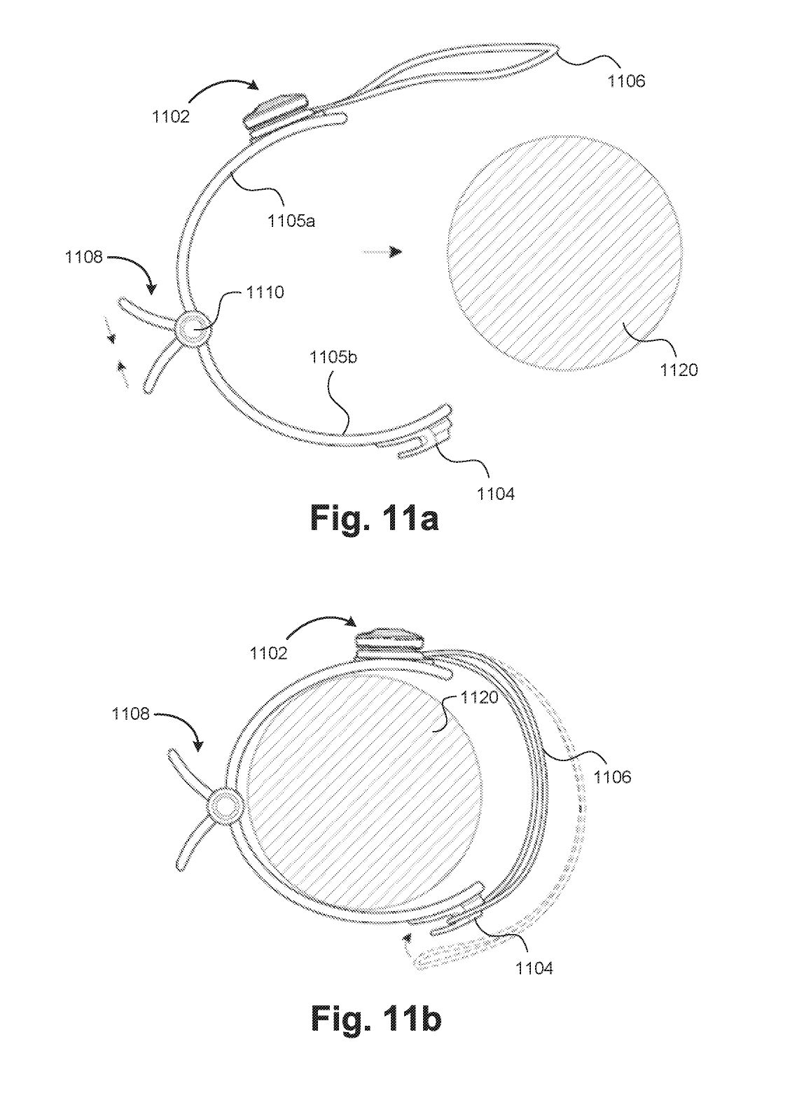

Referring now to FIGS. 11a-b, illustrated is another embodiment of a tourniquet device, which is especially suited for placement around relatively large limbs and which may be ideal for application and use via a single hand. The tourniquet device includes a reel based closure device 1102 that is positioned on a distal end of a first body member 1105a. A lace extends from the reel based closure device 1102 and forms a loop 1106. A guide member 1104 having an open channel is positioned on a distal end of a second body member 1105b. The open channel of the guide member 1104 is configured to allow the lace loop 1106 to be positioned around and within the channel. The first body member 1105a is coupled with the second body member 1105b at a pivot point 1110 that allows the body members to pivot in relation to one another. A pivot mechanism 1108, such as a pair of levers, is positioned near the pivot point 1110. The pivot mechanism 1108 may be employed to cause the first body member 1105a and the second body member 1105b to pivot away from one another, which opens or widens a gap of the tourniquet device.

A limb 1120 may then be easily inserted within the gap of the tourniquet device and the pivot mechanism 1108 may be released to close or narrow the gap with the limb positioned therein. FIG. 11b illustrates the limb 1120 positioned within the gap of the tourniquet device. In some instance, the tourniquet device may include a spring or other biasing component that causes the gap to close or narrow when the pivot mechanism 1108 is released. With the limb 1120 inserted within the tourniquet device, the lace may be wrapped around a portion of the limb and coupled with the guide member 1104 by inserting the loop 1106 within the guide member's channel. The reel based closure device 1102 may then be employed to tension the lace and thereby constrict the first body member 1105a and the second body member 1105b about the limb 1120.

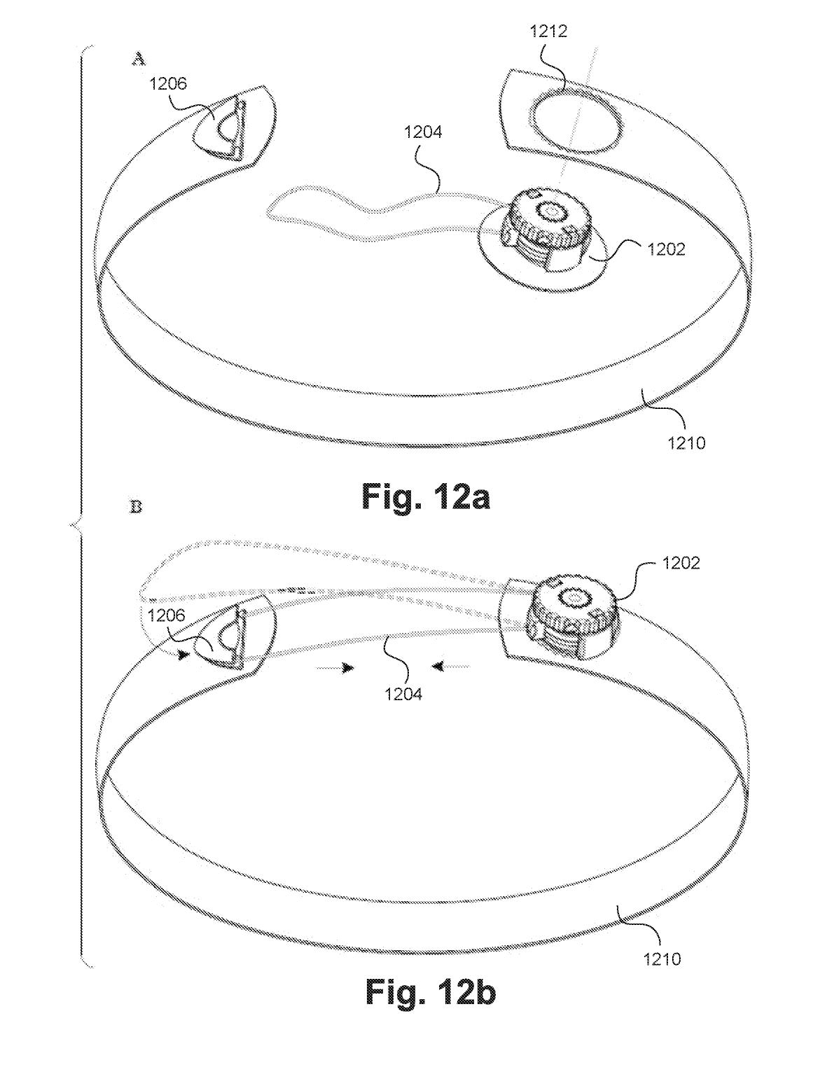

The tourniquet device of FIGS. 12a-b is similar to that illustrated in FIG. 10b in that the reel based closure device 1202 is designed for insertion through a hole 1212 of a strap body 1210 in order to attach the reel based closure device 1202 to the strap body 1210. One of the differences, however, is that the strap body 1210 is a strap component that is specifically designed for use in the tourniquet system rather than being any piece of cloth, such as the fabric of an article of clothing, as in FIG. 10b. The use of the attachable reel based closure device 1202 may allow a user to select an appropriate strap body 1210 for a given application. For example, a wider strap body 1210 may be selected when it is desired to distribute the force across a greater area, while a narrower strap body may be selected when a concentrated force needs to be applied or when only a small segment of the limb is accessible for use with the tourniquet system.

A guide member 1206 is attached to an end of the strap body 1210 opposite the reel based closure device 1202. The guide member 1206 includes an open channel that allows the lace 1204 to be positioned around the guide member 1206 and within the channel as shown in FIG. 12b. The reel based closure device 1202 may then be operated to tension the lace 1204 and constrict the strap body 1210 about a limb as desired. The strap components of FIGS. 12a-b could be located in one or more of the various locations illustrated in FIG. 9. The reel based closure device 1202 could be a separate from the strap component and could be attached to the strap body 1210 based on the injury and/or tourniquet that is needed.

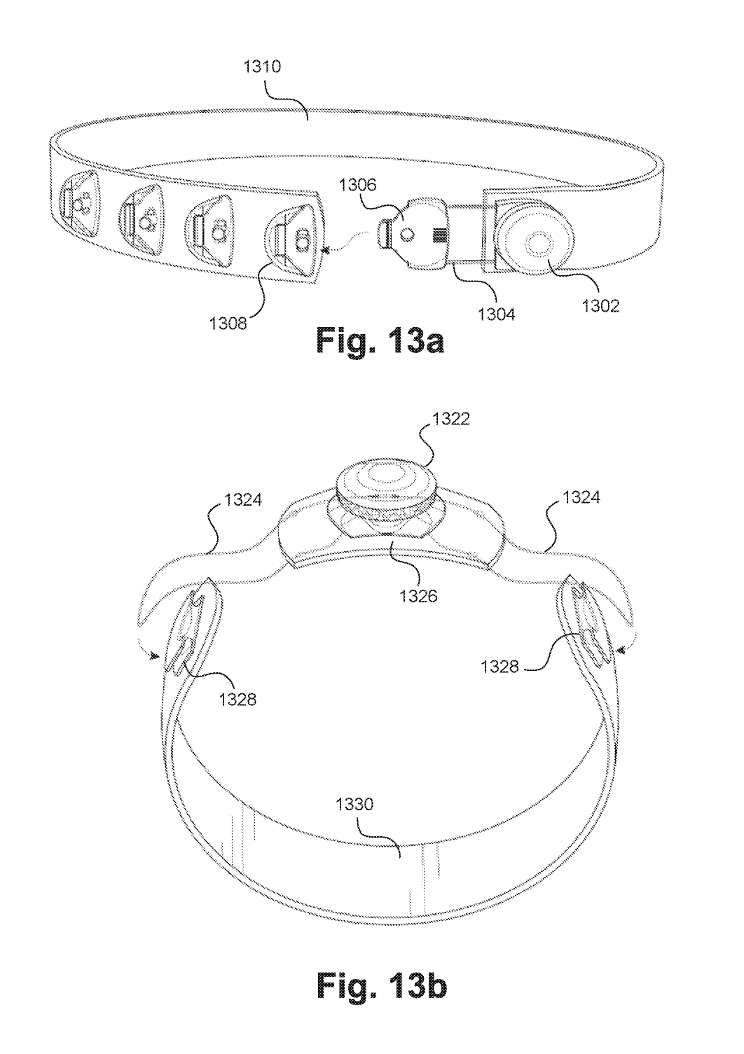

FIG. 13a illustrates a tourniquet device that is configured to enable a macro adjustment of a strap body 1310 about a limb prior to operating a reel based closure device 1302 to tighten the strap body 1310. The macro adjustment feature of the tourniquet device allows the strap body 1310 to be quickly and appropriately positioned around various different sized limbs and does not rely on the reel based closure device 1302 as the sole means of adjusting the size or diameter of the strap body 1310. The macro adjustment feature is provided via a male coupling component 1306 that is attached to the lace 1304 and a plurality of female coupling components 1308 that are positioned circumferentially around and attached to the strap body 1308 in a ladder-like configuration. The male coupling component 1306 may be attached to any of the female coupling components 1308 as desired, which varies the diameter of the strap body 1310. In this manner, the strap body 1310 may be positioned about a limb and the male coupling component 1306 may be attached to a female coupling component 1308 so that an inner surface of the strap body 1310 is positioned against or immediately adjacent the limb. The reel based closure device 1302 may then be employed to further decrease the diameter of the strap body and constrict the strap body against the limb.

FIG. 13b illustrate a tourniquet device that includes a strap body 1330 having a pair of guide members 1328 attached to opposing ends of the strap body 1330. Each of the guide members 1328 includes an open channel that enables a corresponding loop of lace 1324 to be positioned around and within the open channel in order to attach a reel based closure device 1322 with the strap body 1330. Operation of the reel based closure device 1322 tensions the corresponding lace loops 1324, which causes the opposing ends of the strap body 1330 to be pulled toward one another and tighter against a limb positioned within the tourniquet device. In some instances, the reel based closure device 1322 may be positioned on a pad member 1326 that prevents direct contact between the reel based closure device 1322 and the limb. The removable reel based closure device 1322 may allow an appropriately sized or constructed strap body 1330 to be used for a given application. The strap components of FIGS. 13a-b could be located in one or more of the various locations illustrated in FIG. 9 and/or the reel based closure devices could be attached to the strap components based on the injury and/or tourniquet that is needed.

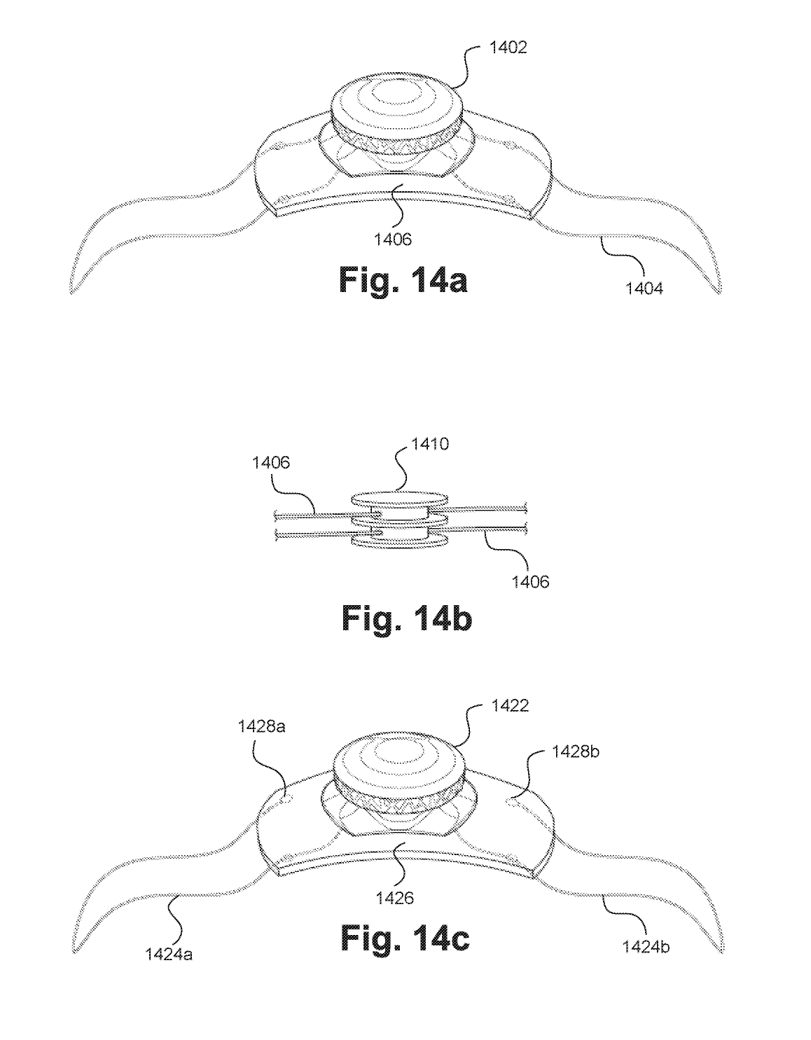

FIGS. 14a-c illustrate variations of the reel based closure devices, lace, and pad members that may be employed in the tourniquet system of FIG. 13b. FIG. 14a illustrates a reel based closure device 1402 that is positioned atop a pad member 1406 and operably coupled with a lace 1404. The lace is typically a single segment of lace with two opposing ends that are attached to a spool within the reel based closure device 1402. The single lace segment 1404 extends as a continuous or unbroken cord between the two opposing ends and forms the two lace loops that are positionable over the opposing guide members of the strap body 1330.

In FIG. 14c the reel based closure device 1422 is coupled with two separate lace segments, 1424a and 1424b. Each lace segment, 1424a and 1424b, includes a proximal end that is attached to a spool positioned within the reel based closure device 1422 and a distal end, 1428a and 1428b, that is attached to the pad member 1426 or reel based closure device 1422. FIG. 14b illustrates a spool member 1410 that may be used to attach lace segments 1406. The spool 1410 include separate lace channels about which the lace segments 1406 may be wound. Operation of the reel based closure device 1422 independently tensions each of the lace segments, 1424a and 1424b. In some instances, the reel based closure device 1422 may be configured to vary the tension in each lace segment, 1424a and 1424b, such as by varying the diameters of the separate channels in FIG. 14b.

While several embodiments and arrangements of various components are described herein, it should be understood that the various components and/or combination of components described in the various embodiments may be modified, rearranged, changed, adjusted, and the like. For example, the arrangement of components in any of the described embodiments may be adjusted or rearranged and/or the various described components may be employed in any of the embodiments in which they are not currently described or employed. As such, it should be realized that the various embodiments are not limited to the specific arrangement and/or component structures described herein.

In addition, it is to be understood that any workable combination of the features and elements disclosed herein is also considered to be disclosed. Additionally, any time a feature is not discussed with regard in an embodiment in this disclosure, a person of skill in the art is hereby put on notice that some embodiments of the invention may implicitly and specifically exclude such features, thereby providing support for negative claim limitations.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the device” includes reference to one or more devices and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. A device for applying a constrictive force about a limb
a reel based closure device including:
a housing having an interior region;
a spool that is positioned within the interior region of the housing, the spool being rotatable in a tightening direction within the housing and being rotatable in a loosening direction within the housing;
a load holding mechanism that is configured to enable the spool to rotate within the housing in the tightening direction and to prevent the spool from rotating in the loosening direction; and
a knob that is coupled with the housing and operationally coupled with the spool so that rotation of the knob relative to the housing causes the spool to rotate in the tightening direction within the housing;
a tension member that is coupled with the spool so that the tension member is wound about the spool when the spool is rotated in the tightening direction and so that the tension member is unwound from about the spool when the spool is rotated in the loosening direction; and
a force application component that is positionable about the limb and that is coupled with the tension member so that as the tension member is wound about the spool, the force application component constricts about the limb and thereby applies a constrictive force to the limb.

2. The device of claim 1, wherein the force application component comprises a strap.

3. The device of claim 2, wherein a distal end of the strap is coupled with the tension member and a proximal end of the strap is coupled with the housing of the reel based closure device.

4. The device of claim 3, wherein the tension member is releasable from the distal end of the strap.

5. The device of claim 3, wherein the distal end of the strap includes a guide component that is configured to route or direct the tension member about the distal end of the strap.

6. The device of claim 1, wherein the force application component includes a guide that is configured to route or direct the tension member about the force application component.

7. The device of claim 1, wherein the force application component includes a lock mechanism that engages with the tension member to prevent movement of the tension member relative to the force application component.

8. The device of claim 7, wherein the lock mechanism comprises a pair of cam members.

9. The device of claim 1, wherein the load holding mechanism is disengageable to enable the spool to rotate in the loosening direction.

10. A method of applying a constrictive force about a limb, the method comprising:
positioning a constrictive device about the limb, the constrictive device comprising:
a reel based closure device that includes:
a housing;
a spool that is rotatable in a tightening direction relative to the housing and that is rotatable in a loosening direction relative to the housing;
a load holding mechanism that enables the spool to rotate in the tightening direction and that prevents the spool from rotating in the loosening direction; and
a knob that is operationally coupled with the spool so that rotation of the knob relative to the housing causes the spool to rotate in the tightening direction;
a tension member that is coupled with the spool so that the tension member is wound about the spool when the spool is rotated in the tightening direction and so that the tension member is unwound from about the spool when the spool is rotated in the loosening direction; and
a force application component that is positionable about the limb and that is coupled with the tension member so that as the tension member is wound about the spool, the force application component constricts about the limb; and
rotating the knob to rotate the spool in the tightening direction and wind the tension member about the spool and thereby apply the constrictive force to the limb via the force application component.

11. The method of claim 10, wherein the force application component comprises a strap.

12. The method of claim 11, wherein a distal end of the strap is coupled with the tension member and a proximal end of the strap is coupled with the housing of the reel based closure device.

13. The method of claim 12, further comprising releasing the tension member from the distal end of the strap.

14. The method of claim 12, wherein the distal end of the strap includes a guide component that is configured to route or direct the tension member about the distal end of the strap.

15. The method of claim 10, wherein the force application component includes a guide that is configured to route or direct the tension member about the force application component.

16. The method of claim 10, wherein the force application component includes a lock mechanism that engages with the tension member to prevent movement of the tension member relative to the force application component.

17. The method of claim 16, wherein the lock mechanism comprises a pair of cam members.

18. The method of claim 10, further comprising disengaging the load holding mechanism to enable the spool to rotate in the loosening direction.
